(19) United States
(12) Patent Application Publication
(10) Pub. No.: US 2015/0338399 A1
(43) Pub. Date: Nov. 26, 2015

(54) METHODS FOR DIAGNOSING AND MONITORING DISEASE BY DIRECTLY QUANTIFYING DISEASE MODIFIED BIOMOLECULES

(71) Applicant: VIRACOR-IBT LABORATORIES, Lee's Summit, MO (US)

(72) Inventors: Jianrong LOU, Boyds, MD (US); Brad STEWART, Gaithersburg, MD (US)

(21) Appl. No.: 14/427,442

(22) PCT Filed: Dec. 5, 2013
(86) PCT No.: PCT/US2013/073306

§ 371 Date: Mar. 11, 2015

Related U.S. Application Data
(60) Provisional application No. 61/735,320, filed on Dec. 10, 2012.

(51) Int. Cl. G01N 33/543

(52) U.S. Cl. CPC G01N 33/543; G01N 2333/47; G01N 2800/102

(57) ABSTRACT

Assays for diagnosing or monitoring a disease of interest are provided. The assays detect disease modified bjomolecules (DMBs) in a direct manner by the sequential use of agents with differing specificities. In an exemplary embodiment, the agents are antibodies and the first antibody is specific for a biomolecule that may be modified during the course of 10 the disease, and detects such biomolecules, whether modified or not The second antibody detects only biomolecules that have been modified.

### BACKGROUND OF THE INVENTION

1. Field of the Invention

The invention generally relates to the detection of disease modified biomolecules (DMBs) in order to diagnose or monitor a disease, or to monitor the efficacy of therapeutic treatments. In particular, the invention provides assays which directly detect the presence of DMBs using sequential antibodies with different but complementary binding specificities.

2. Background of the Invention

Detection and confirmation of disease at an early stage and the ability to accurately and systematically monitor disease status thereafter often result in improved clinical outcomes. Unfortunately, in many instances, it is necessary for overt disease symptoms to develop before a diagnosis can be made or confirmed, and observation of gross symptoms is currently often the only practical way to determine the efficacy of treatment protocols. However, by the time overt symptoms appear, irreversible damage to a subject's health may have already taken place. In addition, readily observable symptoms are often common to many different diseases, and even some detectable disease “biomarkers” have been found to be associated with more than one disease, and/or to be present in apparently healthy individuals. These factors negatively impact the ability of health care professionals to provide reliable, specific diagnoses at early stages of a disease, and hamper their ability to successfully provide early intervention and to make successful adjustments to treatment protocols.

Cancer, cardiovascular disease, diabetes, progressive neurological disorders, infectious diseases and autoimmune diseases are all examples of the many disorders for which early detection, confirmation and monitoring are highly desirable. For example, among more than 80 human autoimmune diseases, rheumatoid arthritis (RA) is one of the most frequent systemic and chronic autoimmune diseases that is often not reliably diagnosed until after irreversible joint damage has already occurred. Rheumatoid arthritis affects 0.5 to 1% of the world population, with more than 2 million RA patients in the United States alone, requiring an estimated 10 million doctor visits each year. RA is characterized by joint swelling and joint tenderness at the early stage, leading to destruction of synovial joints and permanent disability. Sadly, as many as 30% of RA patients have to give up their work within three years of diagnosis.

Although the etiology of RA has not been identified, the treatment of RA has made significant progress in the last decade. DMARDs (disease modified anti rheumatic drugs) such as methotrexate and new biologic drugs such as monoclonal antibodies against TNFα and IL6 have dramatically improved disease management and the quality of life for RA patients. One critical aspect to obtain clinical remission with RA and to avoid permanent bone damage in RA patients is early therapeutic intervention. However, the diagnosis of RA, especially in the early stages of disease, has unfortunately proven to be challenging.

The American College of Rheumatology (ACR) and the European League Against Rheumatism (EULAR) have established guidelines for RA disease classification. The 2010 ACR/EULAR criteria include clinical observations such as numbers of joints with morning stiffness and swelling, serological tests of rheumatoid factor (RF) and anti-citrullinated protein antibodies (ACPA), and acute phase reactant testing on ESR (erythrocyte sedimentation rate) and CRP (C-reactive protein). Unfortunately, the early clinical symptoms of RA are similar to those of other types of arthritis and a specific diagnosis of RA usually is possible only 2 to 5 years after the actual onset of disease. However, radiological data shows that usually significant bone destruction in joints has already occurred by that time, typically taking place within the first two years of disease onset. Such joint damage is irreversible. Thus, early, definitive diagnosis of RA is vital for preventing irreversible joint and tissue damage and for successful overall management of the disease.

Several types of autoantibodies are present prior to, or along with, RA clinical symptoms and they can be readily detected in the plasma samples from people with RA. RF is an auto antibody against the constant region of immunoglobulins of the IgG subclass and its presence in plasma samples is easily detected by immunological methods. RF can be detected in about 50-80% of RA patients, but its major limitation as a diagnostic test is that RF is not a specific biomarker for RA. RF can be detected in high percentages in other inflammatory conditions and even in some apparently healthy individuals.

Research in the last decade has shown that post translational modification (PTM) of proteins has a significant role in autoimmune diseases. In RA, citrullination of several native proteins such as vimentin and fibrin has been found in synovial fluid and blood samples. Autoantibodies against these citrullinated proteins may be present within RA patients and anti-citrullinated protein antibody (ACPA) is another important serological biomarker for RA. ACPAs can be measured in several FDA cleared and CE marked tests such as anti-cyclic citrullinated protein (anti-CCP) antibody tests. A large number of clinical trials have demonstrated that anti-CCP assays have high specificity (approximately 95%), but low sensitivity—only being detected in about 50 to 70% of RA patients. In addition, published clinical data show conflicting results as to whether there are links between anti-CCP titers and RA disease activities, and conclusions are largely dependent on the clinical sample sources.

An alternative might be to use proteins with post translational modification (PTM) as disease biomarkers. Post translational modification is an important cellular regulation mechanism, and is in general highly controlled in the cell, However, aberrant modifications of proteins which are not subject to PTM under normal health conditions (e.g. citrullination of vimentin), has been implicated in disease pathogenesis. The generation of antibodies specific to both a protein of interest and a specific post translationally modified form of the protein, while appealing, has been a challenge, and in many cases has been limited to discovery and basic research. For example, several analytical techniques have been used to identify and quantify citrullinated proteins from synovial fluid samples or plasma. These advanced technical tools include two-dimensional (2D) gel electrophoresis, which separates proteins based on the protein's size and charge, and mass spectrometry, which has become a powerful tool for protein identification. Although these advanced analytical tools play a significant role in discovering proteins with post translational modifications, commercial products using these techniques in clinical laboratories are not currently practical and potentially not possible.

Due to a lack of assay sensitivity and/or specificity, as well as to the lack of practical clinical applicability, currently available testing methods have thus far failed to provide reliable, independent and definitive diagnostic assays for diseases caused or characterized by the presence of PTM proteins.

### SUMMARY OF THE INVENTION

The present invention provides assay methods for the diagnosis of diseases by detecting one or more biomolecules that are post-translationally modified in association with a disease and which are therefore characteristic or indicative of the disease. Such biomolecules may be referred to herein as “disease modified biomolecules” or “DMBs”. According to the invention, agents with differing specificities are used sequentially to directly detect, in a biological sample from a subject, one or more DMBs that are characteristic of a disease of interest. This detection strategy is in contrast to prior art assays which are instead based on indirect detection methods which, for example, measure autoantibodies against DMBs but not the DMBs themselves. Data provided herein show that the methods of the present invention are equally specific and significantly more sensitive than prior art assays. The methods utilize at least two agents capable of interacting or reacting with a biomolecule that is capable of being (susceptible to being) modified in a detectable manner, when an associated disease of interest is present in a subject. The presence of the biomolecule in a modified form (i.e. a DMB) is thus indicative of the presence of the disease in the subject, so that, in general, if a statistically relevant amount of a DMB is detected in a suitable sample from a subject, then the subject is deemed to have the disease.

The first agent is capable of reacting with (e.g. binding to) the biomolecule of interest in both its modified and umodified forms. Thus, when a sample is exposed to the first agent, which may be an antibody, the first agent reacts with (e.g. binds) the biomolecule of interest in the sample, whether modified or not. Unreacted (e.g. unbound) extraneous biomolecules that are not of interest are then removed from the sample, and what is left behind is a pool that comprises both modified DMBs and unmodified biomolecules of interest, both of which were sequestered or retained by the first agent. The pool of DMBs and unmodified biomolecules is then exposed to a second agent, which is either directly or indirectly detectable. Significantly, the second agent reacts only with modified forms of the biomolecule, i.e. reacts only with DMBs. Therefore, removal of unreacted second agent from the reaction mixture either 1) leaves behind detectable amounts of the second agent, indicating that DMBs were present in the pool, and hence in the original sample; or 2) leaves behind no detectable second agent, indicating that DMBs were not present in the pool. This information allows a practitioner to conclude that the subject does have the disease of interest (if DMBs are detected) or does not have the disease of interest (if DMBs are not detected).

Alternatively, the first agent may be capable of reacting with a particular type of modification such as citrullination. When a sample is exposed to this type of first agent, all proteins in the sample with the modification are detected. Then, in a second step, a second agent that is specific for reacting with (e.g. binding to) a particular protein of interest is used to ask whether the protein of interest is among the modified proteins that were detected.

In some embodiments, the method also includes a step or steps of detecting the presence, in the sample, of differentially expressed biomolecules.

Provided herein is a method for diagnosing a disease in a subject, said disease being characterized by the presence of at least one biomolecule that is modified when said disease is present. In some embodiments, the method comprises the steps of: exposing a biological sample obtained from said subject to a first agent that is specific for binding both disease modified forms of said at least one biomolecule and unmodified forms of said at least one biomolecule; forming a pool comprising said disease modified forms of said at least one biomolecule and unmodified forms of said at least one biomolecule by separating biomolecules that were bound by said first agent from biomolecules that were not bound by said first agent in said exposing step; exposing said pool to a second agent that is specific for binding said disease modified forms of said at least one biomolecule, wherein said first and second agents are different from each other and bind different sites on said disease modified forms of said at least one biomolecule; determining an amount of said second agent bound to said disease modified forms of said at least one biomolecule; and diagnosing the presence or absence of said disease in said subject based on said amount of said second agent determined in said determining step. In some embodiments, the method further comprises the step of establishing the identity of said at least one biomolecule that is modified when said disease is present, prior to said step of exposing. In some embodiments, the first agent is a first antibody and said second agent is a second antibody. The second antibody may comprise a detectable label and then said step of determining includes a step of measuring an amount of said detectable label, such as alkaline phosphatase. In some embodiments, the at least one biomolecule is a protein, polypeptide or peptide that is susceptible to disease-dependent citrullination, said first antibody specifically binds said protein, polypeptide or peptide, and said second antibody specifically binds citrullinated residues in proteins, polypeptides and peptides. In other embodiments, the protein, polypeptide or peptide is selected from the group consisting of vimentin, fibrinogen, and alpha enolase, or proteolytic fragments thereof. In yet other embodiments, the disease is rheumatoid arthritis. In further embodiments, the method comprises a step of detecting in said sample at least one biomolecule that is differentially expressed, for example, a nucleic acid such as mRNA, siRNA, miRNA and antisense RNA. In other embodiments, the step of diagnosing includes a step of comparing said amount of said disease modified forms of said at least one biomolecule with an amount of said disease modified forms of said at least one biomolecule which is representative of one or both of i) subjects afflicted with said disease, and ii) subjects not afflicted with said disease. In other embodiments, the step of diagnosing includes determining a percentage of disease modified forms of said at lest one biomolecule in said pool.

Also provided herein is a method for monitoring disease progression or efficacy of a disease treatment in a subject in need thereof, said disease being characterized by the presence of at least one biomolecule that is modified when said disease is present. In some embodiments, the method comprises the steps of: exposing a biological sample obtained from said subject to a first agent that is specific for binding both disease modified forms of said at least one biomolecule and unmodified forms of said at least one biomolecule; forming a pool comprising said disease modified forms of said at least one biomolecule and said unmodified forms of said at least one biomolecule by separating biomolecules that were bound by said first agent from biomolecules that were not bound by said first agent in said exposing step; exposing said pool to a second agent that is specific for binding said disease modified forms of said at least one biomolecule, wherein said first and second agents are different from each other and bind different sites on said disease modified forms of said at least one biomolecule; determining an amount of said second agent bound to said disease modified forms of said at least one biomolecule; and diagnosing the presence or absence of said disease in said subject based on said amount of said second agent determined in said determining step. In some embodiments, the method further comprises the step of establishing the identity of said at least one biomolecule that is modified when said disease is present, prior to said step of exposing. In other embodiments, the first agent is a first antibody and said second agent is a second antibody. The second antibody may comprise a detectable label and said step of determining includes a step of measuring an amount of said detectable label, e.g. alkaline phosphatase. In other embodiments, the at least one biomolecule is a protein, polypeptide or peptide that is susceptible to disease-dependent citrullination, said first antibody specifically binds said protein, polypeptide or peptide, and said second antibody specifically binds citrullinated residues in proteins, polypeptides and peptides. The protein, polypeptide or peptide may be, for example, vimentin, fibrinogen, and alpha enolase, or proteolytic fragments thereof. In further embodiments, the disease is rheumatoid arthritis. In yet other embodiments, the method further comprises a step of detecting in said sample at least one biomolecule that is differentially expressed, for example, a nucleic acid such as mRNA, siRNA, miRNA and antisense RNA. In yet other embodiments, said step of diagnosing includes a step of comparing said amount of said disease modified forms of said at least one biomolecule with an amount of said disease modified forms of said at least one biomolecule which is representative of one or both of i) subjects afflicted with said disease, and ii) subjects not afflicted with said disease. The step of diagnosing may include a step of determining a percentage of disease modified forms of said at least one biomolecule in said pool.

Also provided herein is a method for diagnosing a disease in a subject, said disease being characterized by the presence of one or more types of biomolecules which have a modification of interest when said disease is present. In some embodiments, the method comprises the steps of exposing a biological sample obtained from said subject to a first agent that is specific for binding said modification of interest when present on said one or more types of biomolecules; forming a pool comprising all biomolecules in said sample bound by said first agent; exposing said pool to a second agent that is specific for binding one type of said one or more types of biomolecules; determining an amount of said second agent bound to said one type of said one or more types of biomolecules; and diagnosing the presence or absence of said disease in said subject based on said amount of said second agent determined in said determining step.

Also provided herein is a method for diagnosing a disease in a subject, said disease being characterized by the presence of biomolecules which have a modification of interest when said disease is present. In some embodiments, the method comprises the steps of: determining, in a sample from said subject, a quantity of biomolecules that are susceptible to having said modification of interest; measuring, in said sample, a quantity of biomolecules which have said modification of interest; and diagnosing the presence or absence of said disease in said subject based on said quantity of biomolecules measured in said measuring step. In some embodiments, the steps of determining and measuring are performed by flow cytometry or mass spectrophotometry.

### BRIEF DESCRIPTION OF THE DRAWINGS

FIG. 1A-C. A, schematic representation of an exemplary Direct DMB Assay (DDA assay), in particular a DDA-vimentin (DDA-vim) assay. Anti vimentin=antibody against human vimentin; Anti cit=antibody against citrullinated proteins; AP=Alkaline phosphatase; CDP-STAR=chemiluminescent substrate; B, schematic representation of an alternative embodiment; C, schematic representation of another alternative embodiment.

FIGS. 2A and B. Assay of citrullinated vimentin in a biological sample. A, results (S/N, signal to noise ratio) obtained with a sample (column 1) and controls (columns 2-5), where AP antibody=alkaline phosphatase conjugated goat anti-rabbit IgG antibody; anti Cit=rabbit anti citrullinated protein antibody; Vim=purified human vimentin; anti Vim=goat anti vimentin antibody; B, S/N results for an RA positive sample (Pos) and negative control (NC) and a control assay with no anitcitrulline antibody (No anticit).

FIG. 3. Assay of samples from RA patients and healthy controls. Citrullinated vimentin was detected in RA patients but not in controls. RA, rheumatoid arthritis; AHA, apparently healthy adults; Neg, negative control.

FIG. 4. Citrullinated vimentin test in non RA autoimmune disease samples. Crohn, Crohn's disease; IBD, inflammatory bowel disease; MS, multiple sclerosis; RA, rheumatoid arthritis.

### DETAILED DESCRIPTION

The present invention provides assay methods for the detection, diagnosis and monitoring of diseases of interest. The assays, which may advantageously be used for early stage diagnosis, directly quantify one or more disease modified biomolecules (DMBs) that are associated with or characteristic of a disease of interest. Since the assays are “direct DMB assays”, they may be referred to herein as DDAs. This is in contrast to prior art assays which instead indirectly detect the presence of DMBs, e.g. by detecting autoantibodies against them. Data presented in the Examples section shows that the direct assays of the invention are equally specific and significantly more sensitive than the indirect assays of the prior art. In some embodiments, biomolecules that are differentially expressed in the presence of the disease of interest are also detected and quantitated as part of the assay or in conjunction with the assay.

By “associated with” or “characteristic of” we mean that a particular DMB (and, optionally, over-expressed biomolecule) of interest is present at detectable, statistically significant levels in patients known to have the disease or condition of interest. The presence of the DMB may be caused by the disease or a process associated or driven by the disease (and as such may be considered a symptom of the disease); or the DMB itself may cause one or more symptoms of the disease; or both may be true. Herein, the DMB may be referred to as “associated with” or “characteristic of” a disease, and/or a disease of interest may be described as “associated with” or “characterized by” the presence of one or more types of DMBs. Other equivalent terms/phrases include, for example, “correlated with”, “symptomatic of”, “linked to”, “disease-dependent”, “which cause or which are produced as a result of”; and others which will re readily understood by those of skill in the art. One or more than one DMB may be associated with a disease, and one or more diseases may be associated with a DMB. A “DMB” may refer to a homogenous group of identical molecules, e.g. a particular biomolecule with a consistent chemical formula and stereochemistry, such as a vimentin protein that is always or consistently citrullinated at one particular residue. Alternatively, “a DMB” may refer to category or set or type of DMB, all members of which share a common genre of modification, but for which each member is not necessarily modified in an identical manner. For example, “a DMB” may refer to a group of citrullinated vimentin proteins in which citrulline is present on each protein but not necessarily at the same residue(s) or at the same number of residues. “DMB” may be either singular or plural, as contextually appropriate.

By “biomolecule” we mean a molecule that is found in some, possibly all, living organisms. Biomolecules whose modification is associated with or correlated with the presence of a disease may be of any type known to be modifiable by either enzymatic or non-enzymatic modification, and include but are not limited to: proteins, peptides, nucleic acids (DNA, RNA, DNA/RNA hybrids, etc.), lipids, amino acids, polysaccharides, hormones, vitamins, and other metabolites. The biomolecules may be susceptible to modification in response to or because of disease, or, alternatively, disease symptoms may be caused by modification of the biomolecule, i.e. the presence of the DMB may cause or contribute to the disease. Alternatively, the DMB may be an innocuous side effect of the disease that nevertheless serves as a useful, detectable biomarker.

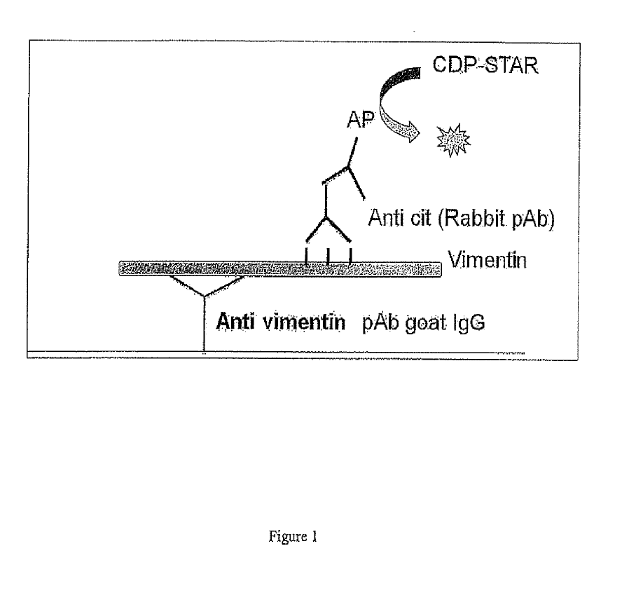

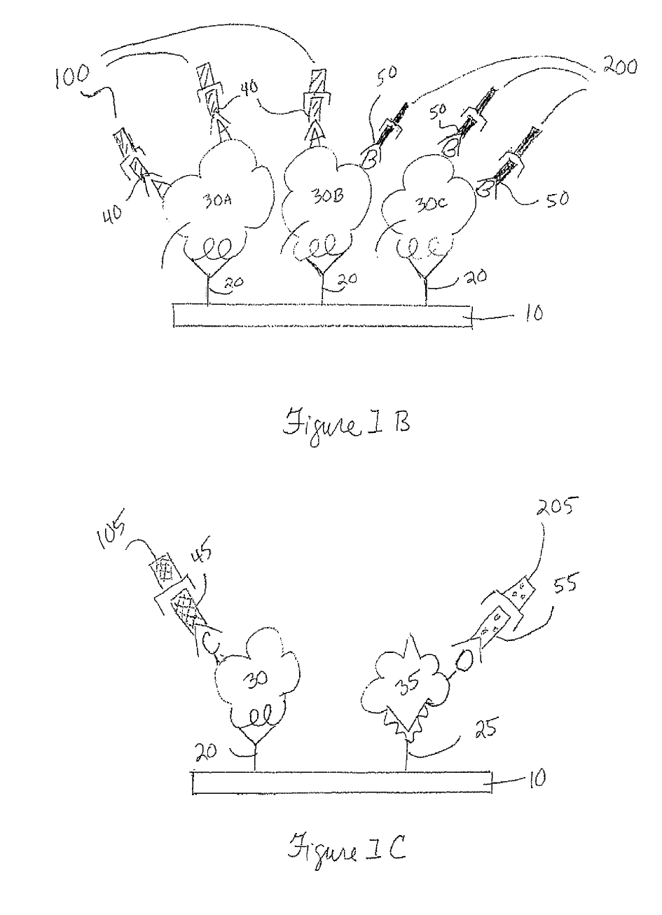

In some embodiments, the method of the invention utilizes at least two agents sequentially (e.g. in tandem, one after the other) to identify the presence of DMBs in a sample of interest, with detection of at least one DMB being indicative of the presence of disease, with the second agent usually being detectable. In some embodiments, the at least two agents are antibodies with different binding specificities. This is illustrated schematically in FIG. 1, which shows an exemplary assay designed to detect citrullinated vimentin in order to diagnose RA (a “DDA_vim” assay). With reference to FIG. 1, a reaction vessel or chamber (e.g. a well of a multi-well plate) is coated with a first “capture” antibody specific for a biomolecule of interest that is known to be subject to disease-associated modification. This antibody is not specific to the relevant disease-associated modification per se, but is capable of binding both disease modified forms of the biomolecule (DMBs) and unmodified forms of the biomolecule (molecules that have not been modified as a result of disease activity).When a biological sample is introduced into the reaction mix, the antibody binds to all forms of the biomolecule which it recognizes, i.e. both modified and unmodified. As a result, a pool comprising modified and unmodified biomolecules is sequestered. As can be seen in FIG. 1, the exemplary biomolecule of interest is vimentin, and an anti-vimentin antibody (depicted as attached to an assay well) has captured a vimentin protein molecule from a biological sample.

In other embodiments of the invention, more than one “first” or “capture” agent may be used, i.e. two or more (multiple, a plurality of, etc.) antibodies, each of which is specific for a different biomolecule of interest, may be employed. Thus, more than one type of biomolecule may be captured in this initial step of the method, and the pool that is sequestered will contain both modified and unmodified forms of each of the types that are present in the sample. Generally, each of the biomolecules is known to be subject to a disease-associated modification of some type, and the different biomolecules may be susceptible to the same or different modifications, e.g. one type of biomolecule may be citrillunated, another may be acetylated, etc.

Next (usually after steps which are well known in the art and thus not described herein in detail e.g. washing or otherwise removing excess agent, sample, etc.), at least one second antibody is added to the well. The second antibody specifically recognizes a disease-induced or associated modification. Such modifications may be changes in sequence, introduction or removal of chemical groups, cleavage or rearrangement of chemical groups, etc., as described more fully below. In the example illustrated in FIG. 1, the modification is citrullination of vimentin, and the second antibody is specifically capable of binding to citrullinated residues. As shown in FIG. 1, the vimentin that was captured by the first antibody is citrullinated and the second antibody binds to sites of citrullination thereon. (Not shown are vimentin molecules that are not citrullinated, but which were also captured by the first antibody, but which are not bound by the second antibody.) In some embodiments, more than one second agent may be employed, i.e. more than one type of disease modification may be detected in the pool, e.g. both citrullination and acetylation of vimentin may be detected, for example, with antibodies that are distinguishable from each other in some manner, so that the amounts of biomolecule with both types of modification can be calculated.

After carrying out steps known to be suitable for immunodiagnostic methods such as those of the invention (e.g. washing to remove excess antibody, etc.), the presence of bound second antibody is detected. In the embodiment illustrated in FIG. 1, this is accomplished by adding a third antibody to the assay wells, the third antibody being capable of binding the second antibody, e.g. an anti-IgG antibody, anti-IgM antibody or an antibody against other antibody fragments. The third antibody is detectable. For example, as shown in FIG. 1, the third antibody may comprise a detectable reagent such as alkaline phosphatase (AP), which is subsequently detected using techniques known in the art, e.g. by exposure to an AP substrate. In this example, detection of AP activity is indicative of the presence in the sample of a DMB, namely, citrullinated vimentin. If multiple second antibodies are employed, then more than one type of detection may be used to render each second antibody separately or individually detectable.

In other embodiments, the second antibodies themselves are directly detectable, i.e. detectable labels as described above for the third antibody are attached directly to the second antibody, making the use of a third antibody unnecessary or optional. However, using a third antibody may provide advantages in certain applications such as for improving assay performance or reducing the assay cost.

The sequential or tandem capture and binding by antibodies directed to two separate epitopes (the first being an epitope of a biomolecule of interest in general, and the second an epitope of a disease-associated modification of the biomolecule of interest), dramatically improves the assay specificity in comparison to prior art assays, without reducing assay sensitivity. Without being bound by theory, it is believed that this may be due to the use of a first antibody to detect particular biomolecules that are possibly, but not necessarily, modified, thereby providing a pool of potentially modified biomolecules that is much smaller and more specific than the pool of all biomolecules, or of all modified biomolecules, in the sample. “Noise” is thus removed from the assay and focused interrogation of highly relevant biomolecules can take place using the second capture antibody, which can only bind a specific modification of interest, i.e. a modification caused by or otherwise associated with the disease.

It is noted that the first and second antibodies generally bind to epitopes at locations or regions of a biomolecule that are sufficiently separated from each other so as to prevent steric interference between binding of the two antibodies. For example, if the modification is citrullination, then the first capture antibody will, in general, bind to an accessible epitope located in or at a region of the biolmolecule that is not susceptible to citrullination. Otherwise, the first antibody might be sterically prevented from binding to biomolecules that had already been citrullinated, and fail to sequester them. Similarly, if proteolytic modification is the PTM that is detected, the first antibody must be directed to an epitope in a region of the protein that is not affected (e.g. removed) by the proteolytic modification, and which does not interfere with the subsequent binding of the second antibody to an epitope characteristic of the proteolytically modified form of the biomolecule.

In some embodiments, one biomolecule of interest (e.g. a protein) may be known to contain or exhibit multiple PTMs, and multiple “second” antibodies may be used to capture all such modified or variant forms of the protein. For example, a protein of interest may be susceptible to both citrullination and deamination, and two types of antibodies, one of which is specific for citrullination and the other of which is specific for deaminated residues, may be employed. Such embodiments are illustrated in FIG. 1B, in which reaction surface 10 is depicted as having three molecules of antibody 20 attached thereto. All 3 antibodies 20 are specific for protein 30 and all three have captured a molecule of protein 30. However, each antibody 20 has captured a protein 30 that is modified in a different way. Protein 30A comprises two sites which contain modification A; protein 30C comprises two sites which contain modification B; and protein 30B has one site comprising modification A and another site comprising modification B. Each of these modification sites can be targeted by a second antibody specific for either A or B, e.g. as shown in FIG. 1B, second antibody 40 is specific for modification A, while second antibody 50 is specific for modification B. In some embodiments, second antibodies A and B may be differentially labeled so as to render them distinguishable from each other. In some embodiments, shown in FIG. 1B, third antibodies may be utilized to distinguish between second antibody 40 and second antibody 50. As can be seen, third antibody 100 is specific for second antibody 40 and third antibody 200 is specific for second antibody 50. Third antibodies 100 and 200 are distinguishable from each other, e.g, by differential labeling or by some other mechanism.

In yet other embodiments, multiple types of “first antibodies” are utilized to capture a mixed pool of different biomolecules, each of which is susceptible to at least one PTM that is different from a PTM of at least one other of the different biomolecules in the pool. In such embodiments, multiple types of “second” antibodies with varying specificities are utilized, and the PTM that is detected by one type of second antibody is generally, although not necessarily always, unique to one type of biomolecule in the mixed pool. Such an embodiment is depicted in FIG. 1C. As shown, first antibody 20, which is specific for protein 30, has captured a molecule of protein 30 from the sample, and first antibody 25, which is specific for protein 35, has captured a molecule of protein 35 from the sample. In the embodiment that is depicted, protein 30 and protein 35 are susceptible to different PTMs: protein 30 is susceptible to modification C and protein 35 is susceptible to modification D. As shown, second antibody 45 is specific for modification C whereas second antibody 55 is specific for modification D. Second antibodies 45 and 55 may be differentially labeled so as to be distinguishable from one another. Alternatively, they may be differentially detected e.g. by exposure to differentially detectable third antibodies 105 and 205, which are specific for second antibodies 45 and 55, respectively.

According to the invention, DMBs are directly detected in biological samples. Biological samples that may be assayed include but are not limited to: synovial fluids, synovial tissues, blood plasma, serum samples, whole blood, urine samples, or tissues, sputum, peripheral blood mononuclear cells (PBMCs), etc.

In some embodiments, the biomolecules that are detected are proteins or polypeptides or peptides that have undergone modification (or, in the case of peptides and polypeptides which have been created, e.g. by cleavage of a larger protein) as a result of PTMs that occur as the result of or in association with at least one disease of interest. Such PTMs include but are not limited to: PTMs involving additions by enzymes such as: addition of hydrophobic groups via myristoylation (attachment of myristate); palmitoylation (attachment of palmitate), isoprenylation or prenylation (the addition of an isoprenoid group (e.g. farnesol—“farnesylation”—and geranyhgeraniol—“geranylgeranylation”); glypiation; etc. PTMs involving addition of cofactors include lipoylation (attachment of a lipoate (C8) functional group; attachment of a flavin moiety (FMN or FAD); heme C attachment via thioether bonds with cysteins; phosphopantetheinylation (the addition of a 4′-phosphopantetheinyl moiety from coenzyme A; retinylidene Schiff base formation; diphthamide formation; ethanolamine phosphoglycerol attachment; hypusine formation; etc. PTMs involving the addition of smaller chemical groups include acylation (e.g. O-acylation (esters), N-acylation (amides), S-acylation (thioesters); acetylation (the addition of an acetyl group, e.g. at the N-terminus of the protein or at lysine residues); formylation; alkylation (addition of an alkyl group, e.g. methyl—“methylation”—, ethyl, etc. usually at lysine or arginine residues); demethylation; amide bond formation (including amidation at C-terminus); amino acid addition such as arginylation, polyglutamylation, and polyglycylation; butyrylation; glycosylation (e.g. the addition of a glycosyl group to arginine, asparagine, cysteine, hydroxylysine, serine, threonine, tyrosine, or tryptophan); polysialylation; malonylation; hydroxylation; iodination; nucleotide addition such as ADP-ribosylation; oxidation; phosphate ester (O-linked) or phosphoramidate (N-linked) formation; phosphorylation (addition of a phosphate group, usually to serine, threonine, and tyrosine (O-linked), or histidine (N-linked)); adenylylation (e.g. addition of an adenylyl moiety to tyrosine (O-linked), or histidine and lysine (N-linked)); propionylation; pyroglutamate formation; S-glutathionylation; S-nitrosylation; succinylation (addition of a succinyl group to lysine); sulfation, the addition of a sulfate group to a tyrosine); selenoylation (co-translational incorporation of selenium in selenoproteins); ubiquitination; neddylation; pupylation; etc.

PTMs involving non-enzymatic additions include but are not limited to glycation (the addition of a sugar molecule to a protein without the controlling action of an enzyme); acylation; pegylation; etc.

PTMs which involve changing the chemical nature of amino acids include but are not limited to: citrullination, or deimination (the conversion of arginine to citrulline); Deamidation (the conversion of glutamine to glutamic acid or asparagine to aspartic acid); Eliminylation (the conversion to an alkene by beta-elimination of phosphothreonine and phosphoserine, or dehydration of threonine and serine); decarboxylation of cysteine; carbamylation (the conversion of lysine to homocitrulline); etc.

PTMs involving structural changes include but are not limited to: formation or disruption of disulfide bridges; proteolytic cleavage at a peptide bond; racemization of proline (by prolyl isomerase); etc.

In some embodiments of the invention, the biomolecule that is modified is a nucleic acid and modification may be defined as, for example, up- or down-regulation of expression or activity of the nucleic acid in response to, or as the result or cause of, a disease of interest; or as a chemical or structural modification of a nucleic acid (such as and various kinds of DNA mutations and DNA methylation).

Diseases that may be diagnosed using the methods of the invention include but are not limited to: various autoimmune diseases such as RA, multiple sclerosis, celiac disease, type 1 diabetes, systemic lupus erythematosus, or any other disease such as cancers with which the production or presence of DMBs is associated or correlated.

Exemplary diseases linked to particular modifications include but are not limited to: RA and citrullinated proteins and/or citrullinated peptides; celiac disease and deamidated gluten peptides or proteins; multiple sclerosis and malondialdehyde modified myelin oligodendrocyte glycoprotein; etc.

In one embodiment, the disease is RA and the DMB is a citrullinated protein,examples of which include but are not limited to, citrullinated vimentin, citrullinated fibrinogen, citrullinated fibronectin, citrullinated collagen II, citrullinated filaggrin, etc. Citrullinated peptides derived from these proteins may also serve as biomarkers for the proposed assay.

Types of disease induced or associated modifications include but are not limited to: chemical modification (e.g. by addition, attachment or incorporation of atoms or molecules not present except when disease is present); cleavage; an increase or decrease in the amount of a biomolecule, i.e. a change in the level of expression of a biomolecule; etc.

Those of skill in the art will recognize that other means/methods of detecting and distinguishing between (or amongst) the second and/or third antibodies may also be used in the practice of the invention, e.g. direct labeling of 2^{nd} antibody with alkaline phosphatase (AP), horse radish peroxidase (HRP) or beta galactosidase, or using 3^{rd} antibodies labeled with enzymes or fluorescent dyes, etc.

The amount of disease modified molecules in a sample may be expressed in a variety of ways that are known to those of skill in the art. For example, the amount may be expressed as an absolute amount or concentration of DMB, based on e.g. correlating the level or amount of signal from a detectable label with known standards. In addition, or alternatively, the amount may be expressed as a percentage or ratio or relative amount of DMBs in the sample, e.g. as a percentage or ratio compared to the amount of DMBs typically found in samples from persons known or believed to be afflicted with the disease of interest, and/or or persons known or believed not to be afflicted with the disease (e.g. apparently healthy individuals). Comparisons may also be made to patients who have previously been treated for the disease, e.g. who have been treated for a particular amount of time or with a particular therapeutic agent or method. Such comparisons may be used to establish the progress of an individual patient in comparison to a “typical” patient that is under treatment. Various scales may also be developed to reflect the values that are measured, e.g. from 0 to 10, where 0 is no DMB present and 10 is the maximum amount detected in a sample; or a scale of 0 to 1000, or any other suitable scale.

When assayed using the methods described herein, samples from patients with the disease of interest have elevated levels of one, or more than one, disease modified biomolecules, while healthy and other disease samples have low or undetectable signals. By “elevated levels” we mean that the quantity of a DMB that is detected in a sample is at least 10% higher or 2 fold, or about 5 fold, or 10 fold or more (e.g. 20, 30, 40, 50, 60, 70, 80, 90, 100, 200, 300, 400, 500, 600, 700, 800, 900 or even 1000-fold, or more) higher than the quantity of a DMB that is detected in control samples from healthy individuals who do not have the disease of interest, when analyzed using relevant statistical methods. Control samples may also be those obtained from patients known to have the disease. One or both of these types of controls may be used to interpret the assay results. For example, if the concentration of DMB A in a healthy population ranges from 100 to 200 pg/ml, a statistically significant cutoff value can be established in order to classify individuals tested using the methods of the invention. If an individual's assay value is statistically significantly less than the cutoff, this individual is not likely to have the disease. On the other hand, if an individual's assay value is statistically significantly higher than the cut off, the individual is likely to have the disease. An individual whose assay value is near the cutoff (e.g. either slightly more or slightly less than the cutoff, e.g. within experimental error), it may be likely that this individual is in the early phase of disease progression.

Similarly, samples from patients in remission may also have lower levels of DMBs, and suitable statistically significant “cut-off” values or ranges may be established for them as well.

Briefly, the assay workflow includes following steps:
1. Sample collection. Plasma, sera or other suitable samples may be prepared from blood collection tubes by standard clinical lab procedures. Synovial tissues or fluids can also be used in the assay.
2. Step 1: The first (capture) antibody against a specific biomolecule of interest known to be modified in response to the disease of interest (e.g. an autoantigen such as human vimentin) is coated on a support surface such as assay wells, magnetic beads, etc. Patient samples are incubated with the first capture antibody, and materials not bound are washed away. Both modified and non-modified biomolecules of interest (e.g. citrullinated and non-citrullinated vimentins) are captured in this step, and the collection of modified and non-modified biomolecules of interest may be referred to herein as a “pool” of biomolecules of interest.
3. Step II: The second antibody, and anti-modification antibody, is added into the assay wells, e.g. anti-citrulline antibody which only binds to citrulline residues that are incorporated in the citrullinated vimentins.
4. Detection step: In some, but not all, embodiments, a detectable third antibody capable of binding the second antibody but not the first capture antibody is introduced into the assay. This third antibody may be directly detectable (e.g. may be attached to a detectable marker) or may be detectable by some other means, e.g. may have a reporter molecule such as an enzyme attached thereto. In other embodiments, the second antibodies are capable of being detected on their own, without the use of a third antibody, as described elsewhere herein.
5. Signal generation step: A substance or means of detecting the third antibody (e.g. an enzyme substrate) is added to assay wells and is converted to a form that provides a detectable signal, which is subsequently measured using a suitable methodology. In other embodiments, a detectable label (such as a fluorescent label) attached to a third antibody is detected (e.g. by observing or interrogating suitable wavelengths of light), to identify and/or distinguish between or among antibodies that are retained in the well.

In alternative embodiments of the invention, the first step of the method involves exposing a biological sample obtained from a subject to a first agent that is specific for binding a modification of interest known to be present on one or more types of biomolecules when a disease of interest is present in a subject. The first agent binds to or captures all biomolecules in the sample that have the modification, e.g. all citrullinated biomolecules. Generally, unbound first agent is then removed as described above, leaving behind (forming) a pool which comprises all biomolecules that were in the sample which were bound by said first agent, i.e. all molecules that comprised the modification (e.g. all citrullinated proteins or peptides). This pool is then exposed to a second agent that is specific for binding one particular type of biomolecule amongst the group of one or more bound biomolecules which have the modification, e.g. a particular protein such as vimentin. The amount of second agent that is bound to said one type of biomolecule of interest serves as an indicator of the amount of the one type of biomolecule in the sample and, by comparing the amount of bound second agent to a suitable reference value, a practitioner of the method can diagnose the presence or absence of the disease in the subject. Those of skill in the art will recognize that the foregoing descriptions of e.g. types of modification, types of modifiable biomolecules, ways to carry out immunological assays, etc. may also generally be applied to this embodiment of the invention. Further, the applications of this embodiment of the technology are similar to those of other embodiments described herein, e.g. to diagnose or monitor a disease, or to monitor the efficacy of therapeutic treatments, etc.

In some embodiments, the assays of the invention are used to detect or diagnose a disease of interest in patients or subjects who are suspected of having the disease. In other embodiments, the assays are used to monitor or track the progress and/or the response to treatment in subjects who have already been diagnosed with the disease. In such embodiments, the steps of the method are essentially the same but may include steps of identifying a patient with the disease, administering a treatment to the patient, and then usually (although not in all embodiments) repeatedly, at suitable spaced-apart time intervals, obtaining and assaying biological samples from the patient as described herein. The results may be interpreted and used by a skilled practitioner (e.g. a physician or other health professional) to assess the patient's state of disease progress, whether or not therapeutic measures should be adopted for the patient (e.g. whether or not medications should be administered), and/or whether or not medications that are being administered are having the desired effect. The ease and accuracy of the assay allows fine tuning of treatment protocols over relatively short time periods, since it is not necessary to wait for manifestations of gross symptoms of the disease to arise or abate. For example, the amount of a medication may be adjusted up or down (increase or decreased), even on a short time scale (e.g. over a period of a few days or weeks), depending on the results obtained with the assay. Or the efficacy of a medication may be evaluated: when there is no positive or desired change in response to a medication, another may be tried instead or in combination with the first medication, or the dose may be increased, etc. If a patient is successfully treated, e.g. so that the assay no longer detects DMBs whereas prior to or earlier in treatment the patient's samples were DMB positive, (or alternatively if the amount of a DMB that is detected decreases to a level that is considered acceptable or manageable) then the patient may be able to forego additional medication. Instead, the patient may be monitored so that future possible needs for treatment can be assessed.

In addition, biologic agents such as TNF inhibitors and IL-6 antagonists have emerged as effective drugs to treat various autoimmune disorders. However, the responses to such biological agents vary greatly across different patient populations. The present technology provides a diagnostic assay which determines whether a patient is responding or is likely to respond to a specific biologic drug.

As described above, in addition to being costly and time consuming, the development of antibodies specific for both protein sequences and disease modified residues within protein sequences has not proven to be particularly practical or successful. However, several modem technologies are emerging that can be used as described herein to directly detect and/or measure disease modified biomolecules. The use of such techniques may circumvent the need to develop antibody recognition techniques and permit the direct analysis or interrogation of biological samples in order to identify and/or quantitate DMBs in the sample. Exemplary techniques include but are not limited to mass spectrophotometry and flow cytometry.

Mass spectrophotometry (MS). Mass spectrophotometry measures the mass/charge ratio of charged biomolecular fragments, and can be used to detect DMBs in biological samples. Peptide fragments with or without modifications can be identified and measured with a variety of mass spectrophotometry technologies such as Matrix-assisted laser desorption/ionization time-of-flight (MALDI TOF) and Liquid chromatography-tandem mass spectrometry (LC MS/MS), Liquid chromatography-selected reaction monitoring mass spectrometry (LC-SRM-MS) or Liquid chromatography-multiple reaction monitoring mass spectrometry (LC-MRM-MS). Target based MS measurements such as SRM and MRM has great sensitivity and specificity to measure DMB in the clinical samples.

Flow cytometry. DMBs in the biological samples can also be analyzed using flow cytometry. For example, unmodified protein sequences or fragments can be bound with labeled antibody and specifically modified residues can be bound with another labeled antibody, and can be distinguished from one another using flow cytometry. Further, the concentration of a DMB of interest and other entities or metabolites of interest (e.g. a type of medication) can measured in a single reaction using flow cytometery.

Accordingly, the invention also provides methods for diagnosing a disease in a subject, the disease being characterized by the presence of biomolecules which have a modification of interest when said disease is present. The methods comprise determining, in a sample from the subject, a quantity of biomolecules that are susceptible to having a modification of interest; measuring the quantity of biomolecules which have the modification of interest in the sample, and diagnosing the presence or absence of the disease in the subject based on the quantity of biomolecules that are measured. Those of skill in the art will recognize that suitable controls or standards are first established as described elsewhere herein, so that the experimental values obtained by the practice of the can be compared in order to establish the diagnosis.

The following examples are intended to illustrate various embodiments of the invention but should not be interpreted so as to limit the scope or spirit of the invention in any way.

### EXAMPLES

### Example 1

A novel assay design (DDA_vim) that Directly Quantifies Citrullinated vimentins from RA patient's plasma

Assay wells were coated with the first capture antibody (pAb goat IgG against human vimentin). An anti-CCP positive plasma sample from an RA patient was added to assay wells and both citrullinated and non-citrullinated vimentins (citrullinated or not) are bound by the first capture antibody. Assay wells were washed to remove excess sample and unbound antibody.

The second capture antibody (pAb rabbit anti-citrulline) was then added to the assay wells. This second antibody selectively binds to citrulline residues such as those that are incorporated into citrullinated vimentins, and thus will bind only to the subset of captured vimentins that are citrullinated, if any are present. Signal detection was accomplished using an alkaline phosphatase (AP) labeled detection antibody specific for binding rabbit IgG, and CDP-STAR® was used as the AP enzyme substrate. Wells with no samples served as negative controls.

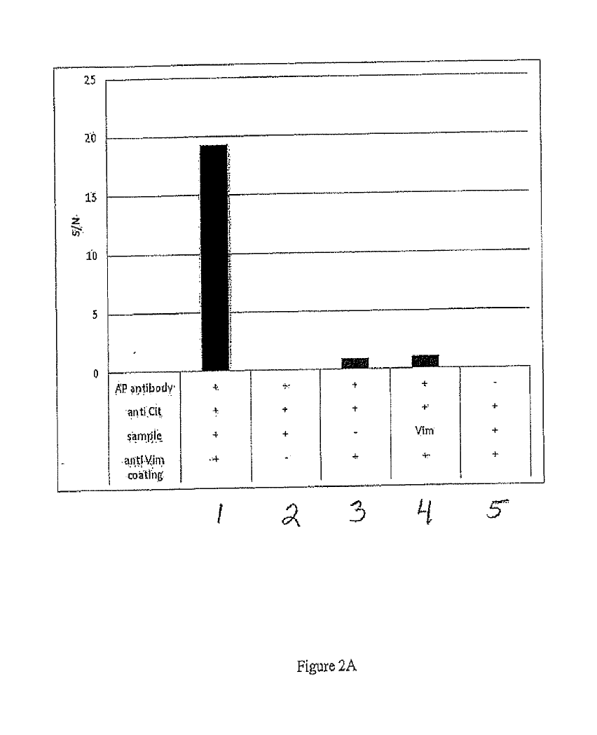

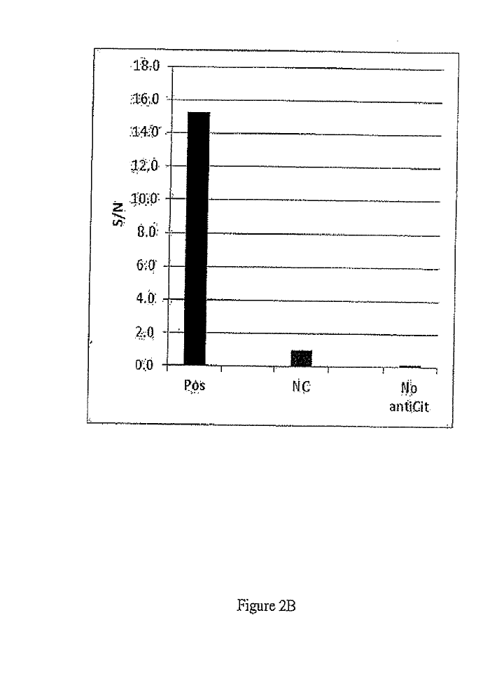

The results are presented in FIG. 2A. As can be seen, positive signal to noise (S/N) was detected in the RA plasma (condition 1), but the signal was completely abolished in wells without the first capture antibody (against human vimentin, condition 2), as well as in wells without the AP labeled detection antibody (condition 5). Wells without samples served as the negative control (condition 3). The assay did not detect purified non-citrullinated vimentins as shown in condition 4. In a similar experiment shown in FIG. 2B, the positive signal was also dependent on the presence of the second capture antibody against citrulline. These experiments demonstrate that this assay design (“DDA-vim”) can specifically detect the presence of citrullinated vimentin in RA patient samples, and can distinguish between the presence of natural non-citrullinated vimentin and citrullinated vimentin.

### Example 2

### Citrullinated Vimentin Detected in Plasma Samples from RA Patients But Not with Apparently Healthy Adults (AHA)

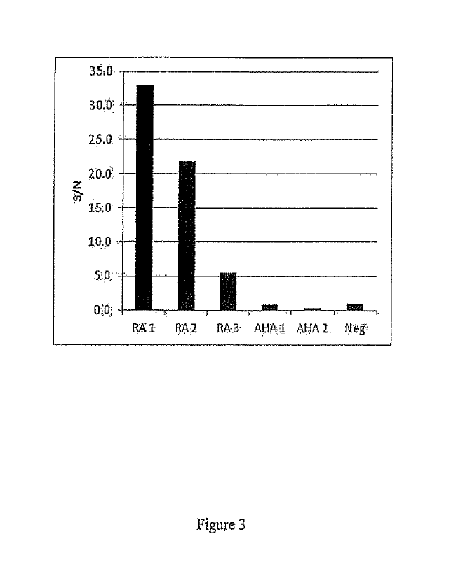

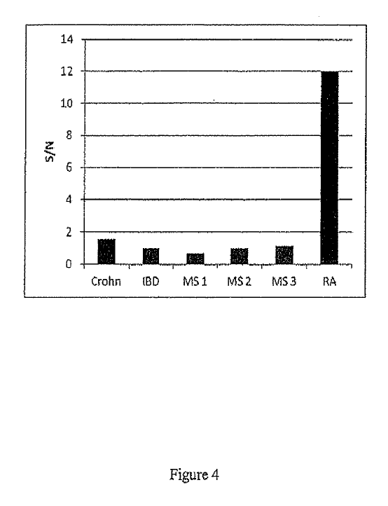

Three RA patient samples and two healthy controls were tested using the assay described in Example 1. The results are shown in FIG. 3. As can be seen, the S/N values ranged from approximately 5 to 32. In contrast, citrullinated vimentin was not detected in 2 healthy controls (FIG. 3) and 5 disease control samples (FIG. 4), which included plasma samples from non-RA autoimmune diseases: Crohn's disease, inflammatory bowel disease (IBD) and multiple sclerosis (MS). As shown in FIG. 4, S/N values from these disease control samples are less than 2.

### Example 3

### Citrullinated Vimentin Testing in Anti-CCP Negative RA Samples

The sensitivity of commercial anti-CCP assays is about 50-70%. Four RA samples that were negative for CCP according to a conventional anti-CCP assay were tested using the DDA-vim assay of the invention. Of the four, two samples were citrullinated vimentin positive according to DDA-vim. As can be seen in Table 1, the two positive samples had S/N values of 2 or higher, indicating the presence of citrullinated vimentin in these samples that were deemed negative by conventional assay methods. In the other two anti-CCP negative samples, both the DDA and anti-RF tests were negative.

This result shows that the novel DDA has improved assay sensitivity compared to commercial anti-CCP assays.

TABLE 1

| RA samples | S/N-Vim | anti-CCP | anti-RF |
|---|---|---|---|
| A101 | 2.0 | 0 | 8 |
| A166 | 18.4 | 0 | 61 |
| PT29 | 1.2 | 0 | 0 |
| PT27 | 1.5 | 0 | 0 |

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for diagnosing a disease in a subject, said disease being characterized by the presence of at least one biomolecule that is modified when said disease is present, comprising:
exposing a biological sample obtained from said subject to a first agent that is specific for binding both disease modified fonns of said at least one biomolecule and unmodified forms of said at least one biomolecule;
fanning a pool comprising said disease modified forms of said at least one biomolecule and unmodified forms of said at least one biomolecule by separating biomolecules that were bound by said first agent from biomolecules that were not bound by said first agent in said exposing step;
exposing said pool to a second agent that is specific for binding said disease modified forms of said at least one biomolecule, wherein said first and second agents are different from each other and bind different sites on said disease modified forms of said at least one biomolecule;
determining an amount of said second agent bound to said disease modified forms of said at least one biomolecule; and
diagnosing the presence or absence of said disease in said subject based on said amount of said second agent determined in said determining step.

2. The method of claim 1, further comprising the step of establishing the identity of said at least one biomolecule that is modified when said disease is present, prior to said step of exposing.

3. The method of claim 1, wherein said first agent is a first antibody and said second agent is a second antibody.

4. The method of claim 3, wherein said second antibody comprises a detectable label and said step of determining includes a step of measuring an amount of said detectable label.

5. The method of claim 4, wherein said detectable label is alkaline phosphatase.

6. The method of claim 3 wherein said at least one biomolecule is a protein, polypeptide or peptide that is susceptible to disease-dependent citrullination, said first antibody specifically binds said protein, polypeptide or peptide, and said second antibody specifically binds citrullinated residues in proteins, polypeptides and peptides.

7. The method of claim 6 wherein protein, polypeptide or peptide is selected from the group consisting of vimentin, fibrinogen, and alpha enolase, or proteolytic fragments thereof.

8. The method of claim 6 wherein said disease is rheumatoid arthritis.

9. The method of claim 1 further comprising a step of detecting in said sample at least one biomolecule that is differentially expressed.

10. The method of claim 9, wherein said at least one biomolecule that is differentially expressed is a nucleic acid.

11. The method of claim 10, wherein said nucleic acid is selected from the group consisting of mRNA, siRNA, miRNA and antisense RNA.

12. The method of claim 1, wherein said step of diagnosing includes a step of comparing said amount of said disease modified forms of said at least one biomolecule with an amount of said disease modified forms of said at least one biomolecule which is representative of one or both of i) subjects afflicted with said disease, and ii) subjects not afflicted with said disease.

13. The method of claim I wherein said step of diagnosing includes determining a percentage of disease modified forms of said at lest one biomolecule in said pool.

14. A method for monitoring disease progression or efficacy of a disease treatment in a subject in need thereof, said disease being characterized by the presence of at least one biomolecule that is modified when said disease is present, comprising:
exposing a biological sample obtained from said subject to a first agent that is specific for binding both disease modified forms of said at least one biomolecule and unmodified forms of said at least one biomolecule;
forming a pool comprising said disease modified forms of said at least one biomolecule and said unmodified forms of said at least one biomolecule by separating biomolecules that were bound by said first agent from biomolecules that were not bound by said first agent in said exposing step;
exposing said pool to a second agent that is specific for binding said disease modified forms of said at least one biomolecule, wherein said first and second agents are different from each other and bind different sites on said disease modified forms of said at least one biomolecule;
determining an amount of said second agent bound to said disease modified forms of said at least one biomolecule; and
diagnosing the presence or absence of said disease in said subject based on said amount of said second agent determined in said determining step.

15. The method of claim 14, further comprising the step of establishing the identity of said at least one biomolecule that is modified when said disease is present, prior to said step of exposing.

16. The method of claim 14, wherein said first agent is a first antibody and said second agent is a second antibody.

17. The method of claim 16, wherein said second antibody comprises a detectable label and said step of determining includes a step of measuring an amount of said detectable label.

18. The method of claim 17, wherein said detectable label is alkaline phosphatase.

19. The method of claim 16 wherein said at least one biomolecule is a protein, polypeptide or peptide that is susceptible to disease-dependent citrullination, said first antibody specifically binds said protein, polypeptide or peptide, and said second antibody specifically binds citrullinated residues in proteins, polypeptides and peptides.

20. The method of claim 19 wherein protein, polypeptide or peptide is selected from the group consisting of vimentin, fibrinogen, and alpha enolase, or proteolytic fragments thereof.

21. The method of claim 19 wherein said disease is rheumatoid arthritis.

22. The method of claim 14 further comprising a step of detecting in said sample at least one biomolecule that is differentially expressed.

23. The method of claim 22, wherein said at least one biomolecule that is differentially expressed is a nucleic acid.

24. The method of claim 23, wherein said nucleic acid is selected from the group consisting of mRNA, siRNA, miRNA and antisense RNA.

25. The method of claim 14, wherein said step of diagnosing includes a step of comparing said amount of said disease modified forms of said at least one biomolecule with an amount of said disease modified forms of said at least one biomolecule which is representative of one or both of i) subjects afflicted with said disease, and ii) subjects not afflicted with said disease.

26. The method of claim 14 wherein said step of diagnosing includes determining a percentage of disease modified forms of said at least one biomolecule in said pool.

27. A method for diagnosing a disease in a subject, said disease being characterized by the presence of one or more types of biomolecules which have a modification of interest when said disease is present, comprising:
exposing a biological sample obtained from said subject to a first agent that is specific for binding said modification of interest when present on said one or more types of biomolecules;
forming a pool comprising all biomolecules in said sample bound by said first agent;
exposing said pool to a second agent that is specific for binding one type of said one or more types of biomolecules;
determining an amount of said second agent bound to said one type of said one or more types of biomolecules; and
diagnosing the presence or absence of said disease in said subject based on said amount of said second agent determined in said determining step.

28. A method for diagnosing a disease in a subject, said disease being characterized by the presence of biomolecules which have a modification of interest when said disease is present, comprising:
determining, in a sample from said subject, a quantity of biomolecules that are susceptible to having said modification of interest;
measuring, in said sample, a quantity of biomolecules which have said modification of interest; and
diagnosing the presence or absence of said disease in said subject based on said quantity of biomolecules measured in said measuring step.

29. The method of claim 28, wherein said steps of determining and measuring are performed by flow cytometry or mass spectrophotometry.
